(19) United States
(12) Patent Application Publication
(10) Pub. No.: US 2026/0151388 A1
(43) Pub. Date: Jun. 4, 2026

(54) PHARMACEUTICAL COMPOSITION FOR HAILEY-HAILEY DISEASE AND USE THEREOF

(71) Applicant: TANABE PHARMA CORPORATION, Osaka (JP)

(72) Inventor: Ryuta SAITO, Osaka (JP)

(73) Assignee: TANABE PHARMA CORPORATION, Osaka (JP)

(21) Appl. No.: 19/323,465

(22) Filed: Sep. 9, 2025

Related U.S. Application Data
(63) Continuation of application No. PCT/JP2025/023149, filed on Jun. 27, 2025.

(30) Foreign Application Priority Data
Jun. 28, 2024 (JP) 2024-104617

(51) Int. Cl. A61K 31/495; A61P 17/00; C07D 295/26

(52) U.S. Cl. CPC A61K 31/495; A61P 17/00; C07D 295/26

(57) ABSTRACT

A pharmaceutical composition for Hailey-Hailey disease contains 5-methyl-2-(1-piperazinyl)benzenesulfonic acid. The compound may be in a form of an anhydrate thereof, a salt thereof, a hydrate or solvate thereof, or a hydrate or solvate of a salt thereof, as an active ingredient. The hydrate is, for example, 5-methyl-2-(1-piperazinyl)benzenesulfonic acid monohydrate.

### CROSS-REFERENCE TO RELATED APPLICATIONS

The present application is a continuation of and claims the benefit of priority to International Application No. PCT/JP2025/023149, filed Jun. 27, 2025, which is based upon and claims the benefit of priority to Japanese Application No. 2024-104617, filed Jun. 28, 2024. The entire contents of these applications are incorporated herein by reference.

### BACKGROUND OF THE INVENTION

### Field of the Invention

The present invention relates to a pharmaceutical composition for Hailey-Hailey disease and a method for treating or preventing Hailey-Hailey disease.

### Description of Background Art

Hailey-Hailey disease is a type of skin disease in which blister-like eruptions (vesicles) frequently appear in intertriginous areas such as the neck, armpits, and groin due to acantholysis of the epidermis (Guidelines for the treatment of familial benign chronic pemphigus 2023, Journal of the Japanese Dermatological Association: 134 (2), 273-287, 2024). The entire contents of this publication are incorporated herein by reference.

### SUMMARY OF THE INVENTION

According to one aspect of the present invention, a pharmaceutical composition for Hailey-Hailey disease includes 5-methyl-2-(1-piperazinyl)benzenesulfonic acid.

According to another aspect of the present invention, a pharmaceutical composition for acantholysis in Hailey-Hailey disease includes 5-methyl-2-(1-piperazinyl)benzenesulfonic acid.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings, wherein:

FIG. 1A is a graph showing a relationship between administration of 5-methyl-2-(1-piperazinyl)benzenesulfonic acid monohydrate and expression of differentiation marker molecules in an in vitro HHD 3D cultured epidermal model in Example 1, with the vertical axis representing the relative gene expression level based on the gene expression level in keratinocytes on Day 0;

FIG. 1B is a graph showing a relationship between administration of 5-methyl-2-(1-piperazinyl)benzenesulfonic acid monohydrate and expression of differentiation marker molecules in an in vitro HHD 3D cultured epidermal model in Example 1, with the vertical axis representing the suppression rate of the increase in gene expression of differentiation markers induced by ATP2C1 siRNA treatment; and

FIG. 2 is a graph showing a relationship between administration of 5-methyl-2-(1-piperazinyl)benzenesulfonic acid monohydrate and intercellular adhesion in an in vitro epidermal intercellular adhesion disorder model in Example 2.

### DETAILED DESCRIPTION OF THE EMBODIMENTS

Embodiments will now be described with reference to the accompanying drawings, wherein like reference numerals designate corresponding or identical elements throughout the various drawings.

In the present specification, the treatment of a disease includes, for example, the meaning of curing the disease, achieving remission of the disease, alleviation of the disease, or suppressing progression of the disease. Further, the treatment of a disease includes, for example, the meaning of treating symptoms caused by the disease (for example, cure, remission, alleviation, or suppression of the symptoms). The prevention of a disease includes, for example, the meaning of preventing the onset of the disease, preventing development of the disease, or preventing recurrence of the disease. Further, the prevention of a disease includes, for example, the meaning of preventing symptoms caused by the disease (for example, preventing occurrence of symptoms or preventing recurrence of symptoms). In the present specification, the treatment or prevention of a disease can also be referred to as, for example, suppression of the disease. Further, the treatment or prevention of symptoms caused by a disease can also be referred to as, for example, suppression of the symptoms caused by the disease.

The composition of the “epidermis” of the skin is typically the stratum corneum, granular layer, spinous layer, and basal layer.

In the present specification, treatment refers to, for example, a procedure administered to a subject diagnosed by a physician as having a disease or symptoms of a disease. Further, in the present specification, prevention refers to, for example, a procedure administered to a subject who has not developed a disease or symptoms of a disease, with the purpose of preventing the onset of the disease or its symptoms. The subject may be, for example, a test subject or participant, and may be a human (patient) or a non-human animal (affected animal). In the present specification, the term “patient” may include, for example, the meaning of an affected non-human animal and may be interpreted as such. Further, when the subject has not developed the disease or its symptoms, the subject may be referred to as a healthy individual (healthy human or healthy non-human animal) with respect to the disease or symptoms of interest.

Hereinafter, the present invention will be described with reference to specific examples. However, the present invention is not limited to these examples. Unless otherwise specified, the embodiments exemplified below can be cross-referenced with one another. Hereinafter, Hailey-Hailey disease is also referred to as HHD.

### Pharmaceutical Composition for HHD

A pharmaceutical composition for HHD according to an embodiment of the present invention is characterized by containing 5-methyl-2-(1-piperazinyl)benzenesulfonic acid. A pharmaceutical composition for HHD according to an embodiment of the present invention is characterized by containing the above compound, and other compositions, conditions, and the like are not particularly limited.

According to a pharmaceutical composition for HHD according to an embodiment of the present invention described above, treatment or prevention of acantholysis can be performed. A pharmaceutical composition for HHD according to an embodiment of the present invention may be used, for example, for the purpose of treatment, for the purpose of prevention, or for the purpose of both treatment and prevention. Hereinafter, in the present specification, the term “treatment/prevention” can be interpreted as meaning treatment, prevention, or both treatment and prevention. According to a pharmaceutical composition for HHD according to an embodiment of the present invention, for example, safe treatment/prevention with few side effects is possible.

In the present specification, hereinafter, 5-methyl-2-(1-piperazinyl)benzenesulfonic acid is referred to as MPBS. The MPBS is not limited in its form and may include anhydride of MPBS (also referred to as MPBS anhydride), salt of MPBS (also referred to as MPBS salt), hydrate of MPBS or hydrate of the MPBS salt (each also referred to as MPBS hydrate), and solvate of MPBS or solvate of the MPBS salt (each also referred to as MPBS solvate), which are collectively referred to as the MPBS compound of the present invention. The MPBS compound included in a pharmaceutical composition according to an embodiment of the present invention may be, for example, the MPBS anhydride, the MPBS salt, the MPBS hydrate, or the MPBS solvate, and may include only one type thereof or two or more types thereof. A pharmaceutical composition according to an embodiment of the present invention preferably contains the MPBS compound as an active ingredient.

The type of the MPBS salt is not particularly limited, and examples thereof include alkali metal salt, alkaline earth metal salt, amphoteric element salt, amine salt, inorganic acid salt, or organic acid salt. Examples of the alkali metal salt include sodium salt and potassium salt; examples of the alkaline earth metal salt include magnesium salt and calcium salt; and examples of the amphoteric element salt include aluminum salt. Examples of the amine salt include lower alkylamine salt such as triethylamine salt; hydroxy lower alkylamine salt such as 2-hydroxyethylamine salt, bis-(2-hydroxyethyl)amine salt, tris(hydroxymethyl)aminomethane salt, or N-methyl-D-glucamine salt; cycloalkylamine salt such as dicyclohexylamine salt; benzylamine salt such as N,N-dibenzylethylenediamine salt; or dibenzylamine salt. Examples of the inorganic acid salt include hydrochloride, hydrobromide, sulfate, or phosphate, and examples of the organic acid salt include fumarate, succinate, oxalate, or lactate.

The type of the MPBS hydrate is not particularly limited, and an example thereof is monohydrate. The type of the MPBS solvate is not particularly limited. A solvent capable of forming the MPBS solvate is not particularly limited, and is, for example, a non-aqueous solvent, and specific examples thereof include alcohols such as methanol, ethanol, or isopropyl alcohol, acetone, ethyl acetate, methylene chloride, or the like.

In a pharmaceutical composition for HHD according to an embodiment of the present invention, the MPBS compound is preferably the MPBS anhydride or the MPBS hydrate, more preferably the MPBS hydrate, and specifically, the MPBS monohydrate.

The MPBS compound is a known compound. The MPBS compound can be synthesized, for example, using the methods described in Japanese Patent Application Laid-Open Publication No. H3-7263, Japanese Patent Application Laid-Open Publication No. H9-221479, European Patent Application Publication No. 390654, European Patent Application Publication No. 779283, U.S. Pat. Nos. 5,053,409, and 5,990,113, and is a compound that can be obtained by a person skilled in the art.

The MPBS compound can be substituted with a compound represented by the following general formula (I) described in International Publication WO 03/011296, or a salt thereof, or a hydrate or solvate of any of these.

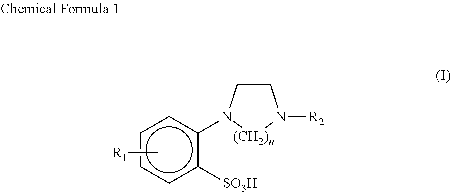

wherein R₁ represents a hydrogen atom, a C₁-C₆ alkyl group, a C₃-C₇ cycloalkyl group, a C₁-C₄ halogenated alkyl group, a halogen atom, or a C₆-C₁₂ aryl group; R₂ represents a hydrogen atom, a C₁-C₆ alkyl group, or a C₇-C₁₂ aralkyl group, the aralkyl group may have one or more substituents selected from a group of a cyano group, a nitro group, a C₁-C₆ alkoxy group, a halogen atom, a C₁-C₆ alkyl group, and an amino group; and n represents an integer from 1 to 4.

In the general formula (I), examples of the C₁-C₆ alkyl group defined by R₁ include methyl, ethyl, propyl, isopropyl, butyl, isobutyl, sec-butyl, tert-butyl, pentyl, isopentyl, neopentyl, tert-pentyl, hexyl, and isohexyl groups. Examples of the C₃-C₇ cycloalkyl group include cyclopropyl, cyclobutyl, cyclopentyl, cyclohexyl, and cycloheptyl groups. Examples of the C₁-C₄ halogenated alkyl group include trifluoromethyl, trifluoroethyl, and pentafluoroethyl groups. Examples of the halogen atom include fluorine, chlorine, and bromine atoms. Examples of the C₆-C₁₂ aryl group include phenyl and naphthyl groups.

Preferred examples of R₁ include, for example, a hydrogen atom, a C₁-C₆ alkyl group, a C₅-C₆ cycloalkyl group, a trifluoromethyl group, a halogen atom, or a phenyl group; more preferred examples include, for example, a C₁-C₃ alkyl group, a cyclohexyl group, a trifluoromethyl group, a chlorine atom, a bromine atom, or a phenyl group; and even more preferred examples include a methyl group or a propyl group, with a methyl group being particularly preferred.

Examples of a C₁-C₆ alkyl group defined by R₂ include the alkyl group defined by R₁ above. Examples of a C₇-C₁₂ aralkyl group include a benzyl group, a phenethyl group, and a naphthylmethyl group. This aralkyl group may have, for example, one or more substituents selected from a group of a C₁-C₆ alkoxy group such as a cyano group, a nitro group, a methoxy group, an ethoxy group, a propoxy group, an isopropoxy group, a butoxy group, an isobutoxy group, a tert-butoxy group, a pentyloxy group, an isopentyloxy group, a tert-pentyloxy group, or a hexyloxy group; a halogen atom as defined by R₁ above; an alkyl group as defined by R₁ above; and an amino group.

Preferred examples of R₂ include, for example, a hydrogen atom, a C₁-C₃ alkyl group, and a C₇-C₁₂ aralkyl group, where the aralkyl group may have one or more substituents selected from a C₁-C₃ alkyl group, a C₁-C₃ alkoxy group, and a halogen atom, and more preferred examples include a hydrogen atom and a C₇-C₁₂ aralkyl group, where the aralkyl group may have one or more substituents selected from a C₁-C₃ alkoxy group, with a hydrogen atom being particularly preferred. Further, in the above general formula (I), n is preferably 2.

In a pharmaceutical composition for HHD according to an embodiment of the present invention, the MPBS compound may be, for example, in an ionized form. When a pharmaceutical composition for HHD according to an embodiment of the present invention is, for example, a liquid as will be described later, and contains an aqueous solvent, a non-aqueous solvent, or a mixed solvent thereof, the MPBS compound may be ionized regardless of whether it is in the form of an anhydride, a salt, a hydrate, or a solvate. The molecular form of the 5-methyl-2-(1-piperazinyl)benzenesulfonic acid (MPBS) is, for example, represented by the following formula (II) and can become an ionized molecular species in a protic solvent such as water. Specifically, examples of the molecular species include a monovalent cation of the following formula (IIIa), a zwitterion having both positive and negative charges of the following formula (IIIb), or a monovalent anion of the following formula (IIIc). The monovalent cation molecular species can, for example, form a salt with an acid (anion), and the monovalent anion can, for example, form a salt with a base (cation).

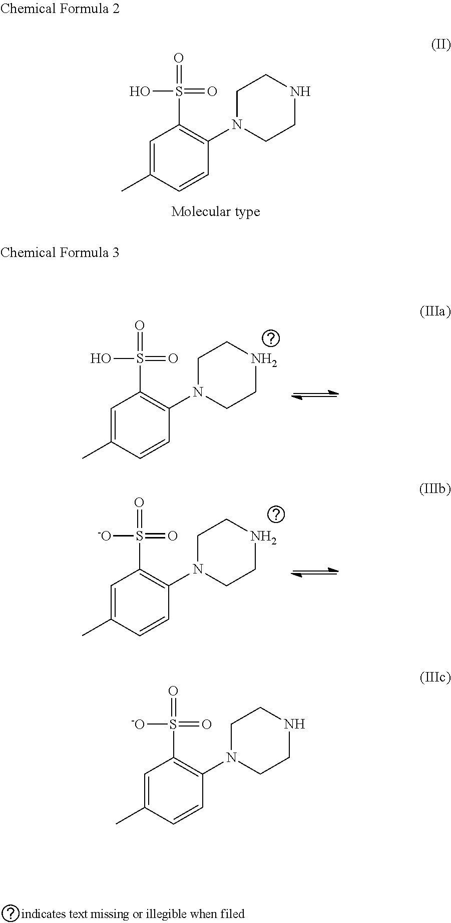

A pharmaceutical composition for HHD according to an embodiment of the present invention can be used, for example, for treatment or prevention of HHD, and the treatment and prevention are, for example, as described above. A pharmaceutical composition for HHD according to an embodiment of the present invention can be interpreted as, for example, a pharmaceutical composition used for symptoms of HHD, and specifically, as a pharmaceutical composition used for treatment or prevention of symptoms of HHD. Further, the treatment or prevention of HHD can also be referred to as, for example, suppression of HHD.

The HHD targeted by the present invention is a disease that causes acantholysis as a pathological symptom. Acantholysis is, for example, a state in the spinous layer of the skin where keratinocytes and the like have lost intercellular adhesion, and can be observed as, for example, clefts or blister formation within the epidermis. Further, acantholytic cells (Tzanck cells), which are floating, round keratinocytes with pyknotic nuclei, can be observed in the blisters. In the skin lesions of HHD patients, for example, abnormal differentiation of keratinocytes is known to cause acantholysis. Acantholysis can be observed, for example, by collecting lesional skin from a patient and examining it according to methods known to a person skilled in the art.

A pharmaceutical composition for HHD according to an embodiment of the present invention can, for example, suppress pathological symptoms of the HHD. The pathological symptoms to be suppressed include, for example, acantholysis, and may further include dyskeratosis in addition to acantholysis. That is, a pharmaceutical composition for HHD according to an embodiment of the present invention may, for example, suppress acantholysis, or suppress both acantholysis and dyskeratosis. According to a pharmaceutical composition for HHD according to an embodiment of the present invention, for example, acantholysis can be suppressed, and both acantholysis and dyskeratosis can be suppressed.

Dyskeratosis refers to, for example, a process in the spinous layer of the skin where keratinocytes individually keratinize to become abnormal keratinocytes, and in the case of HHD, can be observed as granular bodies or acantholytic cells with pyknotic nuclei. Dyskeratosis can be observed, for example, by collecting lesional skin from a patient and examining it according to methods known to a person skilled in the art.

According to a pharmaceutical composition for HHD according to an embodiment of the present invention, for example, the patient's IGA (Investigator's Global Assessment) score can be improved. Here, the IGA score is not particularly limited. The IGA score is generally classified into multiple stages, with higher scores indicating more severe symptoms.

A pharmaceutical composition for HHD according to an embodiment of the present invention can, for example, suppress or improve clinical symptoms of the body caused by the HHD, and specifically, can, for example, suppress or improve at least one selected from a group of lesional skin area, pruritus (itching), pain, and odor in the keratosis. According to a pharmaceutical composition for HHD according to an embodiment of the present invention, for example, by suppressing or improving the above-described pathological symptoms, the clinical symptoms of the body exemplified here can consequently be suppressed or improved.

The lesional skin area is, for example, an area where erythema with clustered vesicles is observed. This erythema is similar to impetigo, where the vesicles have ruptured to form erosions, with the addition of crusts, pustules, pigmentation, or secondary infection. A pharmaceutical composition for HHD according to an embodiment of the present invention can suppress or improve the lesional skin area to, for example, 90% or less, 80% or less, 70% or less, 60% or less, 50% or less, 40% or less, 30% or less, 20% or less, or 10% or less, compared to before administration.

Pruritus can be evaluated, for example, by assessing pruritus felt by a subject using a numerical rating scale for pruritus or a similar method. In the numerical rating scale, for example, 0 indicates no pruritus and 10 indicates the worst pruritus the subject can imagine, and the subject evaluates pruritus using this scale. A pharmaceutical composition for HHD according to an embodiment of the present invention can reduce a post-administration value on the numerical rating scale for pruritus by, for example, 1 or more, 2 or more, 3 or more, 4 or more, 5 or more, 6 or more, 7 or more, 8 or more, or 9 or more, compared to a pre-administration value.

Pain can be evaluated, for example, by assessing the pain felt by a subject using a numerical rating scale for pain or a similar method. In the numerical rating scale, for example, 0 indicates no skin pain and 10 indicates the worst skin pain the subject can imagine, and the subject evaluates skin pain using this scale. A pharmaceutical composition for HHD according to an embodiment of the present invention can reduce a post-administration value on the numerical rating scale for pain by, for example, 1 or more, 2 or more, 3 or more, 4 or more, 5 or more, 6 or more, 7 or more, 8 or more, or 9 or more, compared to a pre-administration value.

Odor can be evaluated, for example, by assessing the odor perceived by a subject using a numerical rating scale for odor or a similar method. In the numerical rating scale, for example, 0 indicates no odor and 10 indicates the worst odor the subject can imagine, and the subject evaluates odor using this scale. A pharmaceutical composition for HHD according to an embodiment of the present invention can reduce a post-administration value on the numerical rating scale for odor by, for example, 1 or more, 2 or more, 3 or more, 4 or more, 5 or more, 6 or more, 7 or more, 8 or more, or 9 or more, compared to a pre-administration value.

A pharmaceutical composition for HHD according to an embodiment of the present invention improves at least one selected from a group of PGIS (Patient Global Impression of Severity), PGIC (Patient Global Impression of Change), CGIS (Clinician Global Impression of Severity), CGIC (Clinician Global Impression of Change), DLQI (Dermatology Life Quality Index), and Skindex-29 in the HHD.
(1) PGIS is a single questionnaire that evaluates a subject's overall impression of severity with a score. A pharmaceutical composition for HHD according to an embodiment of the present invention can improve PGIS and improve the score.
(2) PGIC is a single questionnaire that evaluates a subject's overall degree of improvement in health condition with a score. A pharmaceutical composition for HHD according to an embodiment of the present invention can improve PGIC and improve the score.
(3) CGIS is a single questionnaire that evaluates a physician's overall impression of a subject's severity with a score. A pharmaceutical composition for HHD according to an embodiment of the present invention can improve CGIS and improve the score.
(4) CGIC is a single questionnaire that evaluates a degree of improvement in a subject's overall health condition by a physician with a score. A pharmaceutical composition for HHD according to an embodiment of the present invention can improve CGIC and improve the score.
(5) DLQI is a questionnaire that evaluates each question regarding a subject's QOL with a score. A pharmaceutical composition for HHD according to an embodiment of the present invention can improve DLQI and improve the score.
(6) Skindex-29 is a questionnaire that evaluates each question regarding a subject's QOL with a score. A pharmaceutical composition for HHD according to an embodiment of the present invention can improve Skindex-29 and improve the score.

An administration method of a pharmaceutical composition for HHD according to an embodiment of the present invention is not particularly limited, and may be oral administration or parenteral administration. Examples of parenteral administration include transdermal, subcutaneous, intravenous, intraarterial, intraperitoneal, intranasal, and enteral administration.

A dosage form of a pharmaceutical composition for HHD according to an embodiment of the present invention is not particularly limited, and can be appropriately determined, for example, depending on the administration method. The dosage form is, for example, liquid, gel, cream, or solid. Examples of oral dosage forms include granules, fine granules, powders, tablets, capsules (for example, hard capsules and soft capsules), syrups, emulsions, suspensions, liquids, and jellies. Further, examples of parenteral dosage forms include injections, suppositories, transdermal agents, and the like.

A pharmaceutical composition for HHD according to an embodiment of the present invention need only contain the MPBS compound, and other compositions are not particularly limited. A pharmaceutical composition for HHD according to an embodiment of the present invention may, for example, contain only the MPBS compound as an active ingredient, or may further contain other active ingredients for HHD in addition to the MPBS compound.

A pharmaceutical composition for HHD according to an embodiment of the present invention may, for example, contain only the active ingredient, or may further contain an additive in addition to the active ingredient. The additive is preferably, for example, a pharmaceutically acceptable substance. The type of the additive is not particularly limited, and can be appropriately selected, for example, depending on the dosage form. Examples of the additive include carriers, excipients, stabilizers, lubricants, sweeteners, preservatives, suspending agents, dispersants, thickeners, pH adjusters, antifoaming agents, and flavoring agents. Examples of the carriers include liquids, solids, gels, and creams.

A subject of administration of a pharmaceutical composition for HHD according to an embodiment of the present invention is not particularly limited, and may be, for example, a human or a non-human animal, with a human being preferred. A non-human animal is, for example, a non-human mammal such as a mouse, a rat, a rabbit, or a horse.

Administration conditions of a pharmaceutical composition for HHD according to an embodiment of the present invention are not particularly limited, and can be appropriately determined, for example, depending on the administration method and patient information. The patient information may include, for example, age, sex, body weight, the presence or absence of HHD and its symptoms, the severity of HHD and its symptoms, and medical history.

When a pharmaceutical composition for HHD according to an embodiment of the present invention is administered orally, the following conditions can be exemplified. The following examples can be appropriately adjusted for infants, toddlers, children, adults, elderly patients, or the like according to their patient information and the like. A dose of the MPBS compound can be expressed, for example, as a dose calculated as the MPBS anhydride. The doses exemplified below are doses of the MPBS compound calculated as the MPBS anhydride, and as a specific example, are doses of the MPBS monohydrate calculated as the MPBS anhydride.

### Daily Dose of the MPBS Compound (Amount Calculated as the MPBS Anhydride)

Lower limit: For example, 1 mg, 10 mg, 20 mg, 25 mg, 30 mg, 50 mg, 60 mg, 100 mg, 150 mg, 200 mg, or 300 mg
Upper limit: For example, 300 mg, 400 mg, 500 mg, 600 mg, 800 mg, 1000 mg, or 1200 mg
Range: For example, 1-1000 mg, 10-1000 mg, 20-1000 mg, 25-1000 mg, 30-1000 mg, 50-1000 mg, 60-1000 mg, 100-1000 mg, 1-600 mg, 10-600 mg, 25-600 mg, 30-600 mg, 50-600 mg, 100-600 mg, 10-500 mg, 20-500 mg, 25-500 mg, 30-500 mg, 50-500 mg, 60-500 mg, 100-500 mg, 200-500 mg, 10-400 mg, 20-400 mg, 25-400 mg, 30-400 mg, 50-400 mg, 60-400 mg, 100-400 mg, 200-400 mg, 10-300 mg, 20-300 mg, 25-300 mg, 30-300 mg, 50-300 mg, 60-300 mg, 100-300 mg, 200-300 mg, 10-200 mg, 20-200 mg, 25-200 mg, 30-200 mg, 50-200 mg, 60-200 mg, 100-200 mg, 10-100 mg, 20-100 mg, 25-100 mg, 30-100 mg, 50-100 mg, 60-100 mg, 300-400 mg, 300-500 mg, 300-600 mg, 300-800 mg, 300-1000 mg, 300-1200 mg, 400-600 mg, 400-800 mg, 400-1000 mg, 400-1200 mg, 500-600 mg, 500-800 mg, 500-1000 mg, 500-1200 mg, 600-800 mg, 600-1000 mg, or 600-1200 mg.
Specific examples of the dose: For example, 1 mg, 10 mg, 20 mg, 25 mg, 30 mg, 40 mg, 50 mg, 100 mg, 150 mg, 200 mg, 250 mg, 300 mg, 350 mg, 400 mg, 450 mg, 500 mg, 550 mg, 600 mg, 700 mg, 800 mg, 900 mg, 1000 mg, or 1200 mg.
Number of administrations per day: For example, 1 to 3 times; preferably 1 or 2 times; and more preferably 2 times.
Frequency (interval) of administration: For example, daily.

When a pharmaceutical composition for HHD according to an embodiment of the present invention is administered orally, administration timing can be freely set, for example, before a meal, during a meal, immediately after a meal, after a meal, between meals, when waking up, or when going to bed, with administration between meals being desirable. The inter-meal administration of a pharmaceutical composition for HHD according to an embodiment of the present invention refers to, for example, regarding a time interval from intake of a meal to administration, administration at least 1 hour after, preferably at least 2 hours after intake of a meal, and/or, for example, regarding a time interval from administration to the next meal, the next meal is taken at least 1 hour after, 2 hours after, 3 hours after, 4 hours after, or 4.5 hours after administration, preferably 1 hour after administration. That is, a pharmaceutical composition for HHD according to an embodiment of the present invention is administered, for example, between 1 hour or 2 hours after a meal and 1 hour, 2 hours, 3 hours, 4 hours, or 4.5 hours before the next meal.

As described above, a pharmaceutical composition for HHD according to an embodiment of the present invention can be used for treating or preventing symptoms of HHD, and as described above, HHD is a disease that causes acantholysis. Therefore, an embodiment of the present invention can also be referred to as, for example, a pharmaceutical composition for acantholysis that treats or prevents acantholysis. Further, an embodiment of the present invention can also be referred to as, for example, a pharmaceutical composition for acantholysis and dyskeratosis that simultaneously treats or prevents acantholysis and dyskeratosis.

### Method for Treating or Preventing HHD

A method for treating or preventing HHD according to an embodiment of the present invention includes a process of administering 5-methyl-2-(1-piperazinyl)benzenesulfonic acid to a subject. As described above, the form of 5-methyl-2-(1-piperazinyl)benzenesulfonic acid (MPBS) is not limited and may be any type of the MPBS compound, that is, the MPBS anhydride, the MPBS salt, the MPBS hydrate, and/or the MPBS solvate. Unless otherwise specified, a method for treating or preventing HHD according to an embodiment of the present invention is hereinafter referred to as a treatment/prevention method for HHD according to an embodiment of the present invention. A treatment/prevention method according to an embodiment of the present invention is characterized by ministering the MPBS compound, and other conditions and processes are not particularly limited.

In a treatment/prevention method according to an embodiment of the present invention, the subject is a patient, and the patient may be, for example, a patient who has developed HHD or a patient who has not developed HHD.

In a treatment/prevention method according to an embodiment of the present invention, the administration of the MPBS compound is, for example, administration of a pharmaceutical composition for HHD according to an embodiment of the present invention. In an embodiment of the present invention, the MPBS compound, its composition, its administration method, and the like can incorporate the descriptions regarding a pharmaceutical composition for HHD according to an embodiment of the present invention.

### Use of the MPBS Compound

An embodiment of the present invention is 5-methyl-2-(1-piperazinyl)benzenesulfonic acid for use in treating or preventing HHD. As described above, the form of 5-methyl-2-(1-piperazinyl)benzenesulfonic acid (MPBS) is not limited and may be any type of the MPBS compound, that is, the MPBS anhydride, the MPBS salt, the MPBS hydrate, and/or the MPBS solvate. In an embodiment of the present invention, the MPBS compound, its composition, its method of use, and the like can incorporate the descriptions regarding a pharmaceutical composition for HHD according to an embodiment of the present invention.

Further, an embodiment of the present invention is a use of 5-methyl-2-(1-piperazinyl)benzenesulfonic acid in manufacturing a pharmaceutical composition for HHD. As described above, the form of 5-methyl-2-(1-piperazinyl)benzenesulfonic acid (MPBS) is not limited and may be any type of the MPBS compound, that is, the MPBS anhydride, the MPBS salt, the MPBS hydrate, and/or the MPBS solvate. In an embodiment of the present invention, the MPBS compound, its composition, its method of use, and the like can incorporate the descriptions regarding a pharmaceutical composition for HHD according to an embodiment of the present invention.

### Pharmaceutical Composition for Acantholysis and Its Use

A pharmaceutical composition for acantholysis according to an embodiment of the present invention contains 5-methyl-2-(1-piperazinyl)benzenesulfonic acid. As described above, the form of 5-methyl-2-(1-piperazinyl)benzenesulfonic acid (MPBS) is not limited and may be any type of the MPBS compound, that is, the MPBS anhydride, the MPBS salt, the MPBS hydrate, and/or the MPBS solvate. A pharmaceutical composition for acantholysis according to an embodiment of the present invention may contain, for example, only one type of the MPBS compound, or may contain two or more types of the MPBS compound. The MPBS compound can be used for treating or preventing acantholysis. A pharmaceutical composition for acantholysis according to an embodiment of the present invention is characterized by containing the MPBS compound, and other conditions are not limited in any way. A pharmaceutical composition for acantholysis according to an embodiment of the present invention preferably contains the MPBS compound as an active ingredient. When a pharmaceutical composition for acantholysis according to an embodiment of the present invention is used for treating or preventing acantholysis, the type of disease causing acantholysis as a pathological symptom is not particularly limited.

According to a pharmaceutical composition for acantholysis according to an embodiment of the present invention described above, treatment or prevention of acantholysis can be performed. A pharmaceutical composition for acantholysis according to an embodiment of the present invention may be used, for example, for the purpose of treatment, for the purpose of prevention, or for the purpose of both treatment and prevention. Hereinafter, in the present specification, the term “treatment/prevention” can be interpreted as meaning treatment, prevention, or both treatment and prevention. According to a pharmaceutical composition for acantholysis according to an embodiment of the present invention, for example, safe treatment/prevention with few side effects is possible. An example of a disease in which acantholysis occurs is Hailey-Hailey disease.

In a pharmaceutical composition for acantholysis according to an embodiment of the present invention, the MPBS compound, its composition, and its method of use are not particularly limited, and can, for example, incorporate the descriptions regarding a pharmaceutical composition for HHD according to an embodiment of the present invention described above. Upon incorporation, “HHD” and “symptoms of HHD” can be read as “acantholysis.”

A method for treating or preventing acantholysis according to an embodiment of the present invention includes a process of administering 5-methyl-2-(1-piperazinyl)benzenesulfonic acid to a subject. As described above, the form of 5-methyl-2-(1-piperazinyl)benzenesulfonic acid (MPBS) is not limited and may be any type of the MPBS compound, that is, the MPBS anhydride, the MPBS salt, the MPBS hydrate, and/or the MPBS solvate. Unless otherwise specified, a method for treating or preventing acantholysis according to an embodiment of the present invention is hereinafter referred to as a treatment/prevention method for acantholysis according to an embodiment of the present invention. A treatment/prevention method according to an embodiment of the present invention is characterized by ministering the MPBS compound, and other conditions and processes are not particularly limited. A subject as a target of the present invention is, for example, a patient who has developed or may develop acantholysis as a pathological symptom, and the type of disease that causes acantholysis as a pathological symptom is not particularly limited.

In an embodiment of the present invention, the MPBS compound, its composition, its administration method, and the like are not particularly limited, and can incorporate the descriptions regarding a pharmaceutical composition for HHD according to an embodiment of the present invention. Upon incorporation, “HHD” and “symptoms of HHD” can be read as “acantholysis.”

An embodiment of the present invention is 5-methyl-2-(1-piperazinyl)benzenesulfonic acid for use in treating or preventing antholysis. As described above, the form of 5-methyl-2-(1-piperazinyl)benzenesulfonic acid (MPBS) is not limited and may be any type of the MPBS compound, that is, the MPBS anhydride, the MPBS salt, the MPBS hydrate, and/or the MPBS solvate. In an embodiment of the present invention, the MPBS compound, its composition, and its method of use can incorporate the descriptions regarding a pharmaceutical composition for HHD according to an embodiment of the present invention. Upon incorporation, “HHD” and “symptoms of HHD” can be read as “acantholysis.”

Further, an embodiment of the present invention is a use of 5-methyl-2-(1-piperazinyl)benzenesulfonic acid in manufacturing a pharmaceutical composition for antholysis. As described above, the form of 5-methyl-2-(1-piperazinyl)benzenesulfonic acid (MPBS) is not limited and may be any type of the MPBS compound, that is, the MPBS anhydride, the MPBS salt, the MPBS hydrate, and/or the MPBS solvate. In an embodiment of the present invention, the MPBS compound, its composition, and its method of use can incorporate the descriptions regarding a pharmaceutical composition for HHD according to an embodiment of the present invention. Upon incorporation, “HHD” and “symptoms of HHD” can be read as “acantholysis.”

### EXAMPLES

In the following examples, 5-methyl-2-(1-piperazinyl)benzenesulfonic acid monohydrate (hereinafter may be referred to as Compound A in the examples) was used as the MPBS compound.

### Example 1 Suppression of Abnormal Differentiation in an In Vitro HHD 3D Cultured Epidermal Model

A suppressive effect of Compound A on abnormal differentiation in a HHD 3D cultured epidermal model was evaluated. In the skin lesions of HHD patients, abnormal differentiation of keratinocytes is known to cause acantholysis. In this model, the effect of Compound A on gene expression changes of differentiation markers was investigated.

### Test Cells and Test Design

Since ATP2C1 gene mutations are involved in HHD, an ATP2C1 siRNA-induced in vitro HHD 3D cultured epidermal model was prepared using the following method. Cells and culture medium were obtained using the LabCyte EPI-KIT (Japan Tissue Engineering Co., Ltd., 401810), a human epidermal model preparation kit. Human primary keratinocytes in the human epidermal model preparation kit were transfected with siRNA. For the transfection, Lipofectamine (registered trademark) RNAiMAX Transfection Reagent (Thermo Fisher Scientific, 13778), a cationic lipid reagent, was used as a transfection reagent, and Opti-MEM (registered trademark) Reduced Serum Medium (Thermo Fisher Scientific, 31985-062) was used as a culture medium. As an example, ATP2C1 siRNA (Thermo Fisher Scientific, J-006119-05-0005) was used as the siRNA, and Non-targeting control siRNA (Thermo Fisher Scientific, D-001810-01-05) was used as a control. The final concentration of the siRNA was set to 100 nmol/L. In the present specification, the term “final concentration” regarding solutions of test substances, reagents, and the like refers to the concentration in the culture medium at the final stage after all test substance solutions, reagents, and the like have been added.

Transfected cells into which the siRNA had been introduced were used to initiate three-dimensional culture (Day 0) using inserts for air-liquid interface culture and a 24-well plate, and in vitro epidermal tissue was prepared over 8 days of culture (Day 0 to Day 8). Culture environment conditions were maintained at 37° C. and 5% CO₂. From the start of the three-dimensional culture (Day 0), an aqueous solution of Compound A (solvent: phosphate-buffered saline) was added to the culture medium of the in vitro epidermal tissue to achieve a final concentration of Compound A of 10 μmol/L. The culture medium was replaced daily, and the addition of the Compound A aqueous solution was performed similarly during each replacement. After 8 days (Day 8) since the start of the three-dimensional culture, the in vitro epidermal tissue was sampled and used for evaluation. The study consisted of three groups: a control siRNA-treated group; an ATP2C1 siRNA-treated group; and an ATP2C1 siRNA+Compound A-treated group, which was an example group and was treated with ATP2C1 siRNA and Compound A. The number of samples was set at 9 epidermal tissues per group.

### Evaluation Method

RNA was extracted from the sampled in vitro epidermal tissue using the RNeasy (registered trademark) Mini QIAcube Kit (QIAGEN, 74116). Concentration of the RNA was measured using a microvolume spectrophotometer (DropSense 96 (trademark) (Trinean) or LUNATIC (Unchained Labs)), and then the RNA was reverse-transcribed into cDNA. For the reverse transcription reaction, SuperScript (trademark) IV VILO (trademark) Master Mix (Thermo Fisher Scientific, 11756050) or High-Capacity RNA-to-cDNA (trademark) Kit (Thermo Fisher Scientific, 4387406) was used. Using the cDNA as a template, RT-qPCR analysis was performed using the QuantStudio (trademark) 6 Flex Real-Time PCR System (Applied Biosystems). For the RT-qPCR reaction, TaqMan (registered trademark) Fast Advanced Master Mix (Thermo Fisher Scientific, 4444557) or TaqMan (registered trademark) Fast Universal PCR Master Mix (Thermo Fisher Scientific, 4352042) was used as the master mix, and the TaqMan (registered trademark) probes (Thermo Fisher Scientific) listed in Table 1 were used. As differentiation markers, KRT5 and KRT14 (basal layer markers) and IVL (granular layer marker) were used.

TABLE 1

| Gene | | |
|---|---|---|
| Symbol | Gene Name | Assay ID |
| RPLP0 | ribosomal protein lateral stalk subunit P0 | Hs99999902_m1 |
| HPRT1 | hypoxanthine phosphoribosyltransferase 1 | Hs02800695_m1 |
| KRT5 | keratin 5 | Hs00361185_m1 |
| KRT14 | keratin 14 | Hs00265033_m1 |
| IVL | involucrin | Hs00846307_s1 |

In the RT-qPCR analysis, RPLP0 or HPRT1 was used as the internal reference gene. Then, using the ΔΔCt method, the relative gene expression levels, as well as their average values and standard errors, were calculated based on the average gene expression levels in keratinocytes on Day 0. The suppression rate was calculated as follows.

$$Suppression rate ( \% ) = 100 \times ( A - B ) / ( A - C )$$

A: Average relative gene expression level in the ATP2C1 siRNA-treated group
B: Relative gene expression level in the ATP2C1 siRNA+Compound A-treated group
C: Average relative gene expression level in the control siRNA-treated group

### Statistical Analysis

Statistical analysis was performed using SAS, with the significance level set at two-sided 5%. The success of inducing the in vitro HHD 3D cultured epidermal model was determined by a two-group comparison between the control siRNA-treated group and the ATP2C1 siRNA-treated group, and the pharmacological effect of Compound A was determined by a two-group comparison between the ATP2C1 siRNA-treated group and the example group (ATP2C1 siRNA+Compound A-treated group). In both cases, Student's t-test was used as the test method.

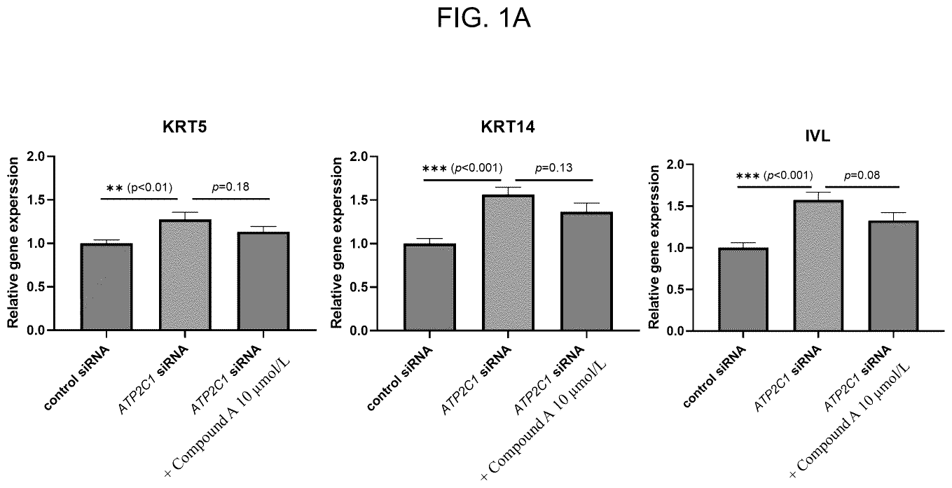

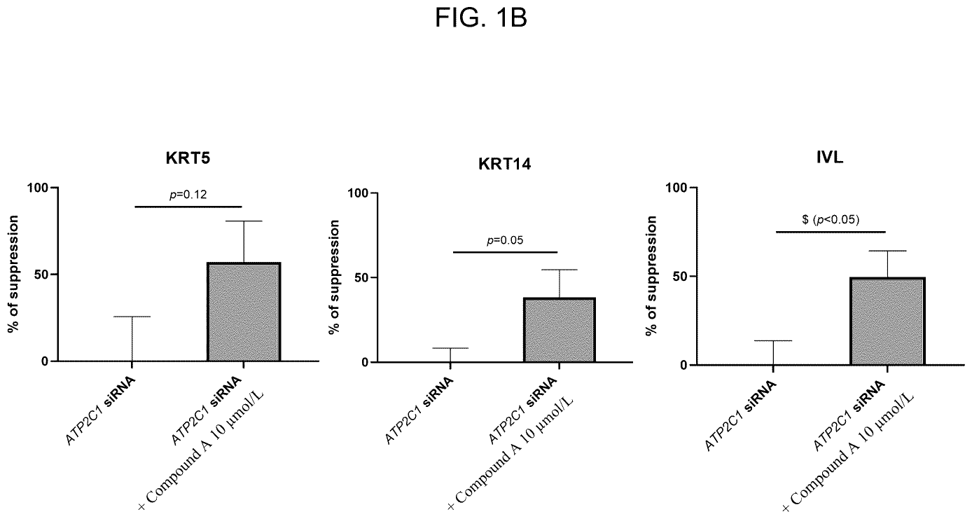

FIGS. 1A and 1B are graphs showing the results of the gene expression analysis, specifically illustrating a relationship between the administration of the MPBS monohydrate and the expression of differentiation marker molecules in the in vitro HHD 3D cultured epidermal model. The vertical axis of FIG. 1A represents the relative gene expression level based on the gene expression level in keratinocytes on Day 0. The vertical axis of FIG. 1B represents the suppression rate of the increase in gene expression of differentiation markers induced by ATP2C1 siRNA treatment.

In the ATP2C1 siRNA-treated group, statistically significant increases in expression were observed for the basal layer markers KRT5 and KRT14, as well as the granular layer marker IVL, which is reported to exhibit abnormal expression in HHD patients, compared to the control siRNA-treated group. In contrast, in the example group, the addition of Compound A suppressed the increased expression of these differentiation markers, correcting the abnormal expression of these marker genes.

### Example 2 Improvement of Intercellular Adhesion Disorder in an In Vitro Epidermalintercellular Adhesion Disorder Model

The effect of Compound A on improving intercellular adhesion was evaluated in an epidermal intercellular adhesion disorder model. Disorder of intercellular adhesion leads to acantholysis. The disorder of intercellular adhesion was evaluated based changes in electrical resistance (Cell Index) using a real-time cell analyzer.

### Test Cells and Test Design

An in vitro epidermal intercellular adhesion disorder model was prepared using 2,2′-methylenebis(6-t-butyl-4-methylphenol) (hereinafter referred to as bisphenol) according to the following method. Bisphenol has been reported to selectively inhibit the ATPase activity of SPCA1, the molecule responsible for HHD. Human primary keratinocytes derived from a White female (KURABO, KK-4109) were used as cells. For the culture of the human primary keratinocytes, HuMedia-KG2 medium (KURABO, KK-2150S) was used. The xCELLigence real-time cell analyzer (Agilent) was used as the real-time cell analyzer and was placed in an incubator under conditions of 37° C. and 5% CO₂.

First, 50 μl/well of HuMedia-KG2 medium was added to an E-Plate VIEW 96 PET (Agilent) plate. The plate was set in the real-time cell analyzer to measure the background electrical resistance value. Subsequently, the plate was removed, and 100 μL/well of the human primary keratinocytes was seeded onto the plate at a density of 25,000 cells/well, and the plate was set again in the real-time cell analyzer. Then, simultaneously with the setting of the plate, measurement of electrical resistance values was started and recorded at 15-minute intervals (Day 0).

On Day 1, the plate was removed. After removing 50 μl/well of culture supernatant from the wells of the plate, a calcium solution was added at 100 μl/well to achieve a final calcium concentration of 1.2 mmol/L. The plate was re-set in the real-time cell analyzer, and measurement of electrical resistance values was resumed. Next, on Day 2, the plate was removed. After removing 100 μl/well of culture supernatant from the wells of the plate, Compound A was added at 50 μl/well. The plate was re-set in the real-time cell analyzer, and measurement of electrical resistance values was resumed. Then, three hours after the addition of Compound A, the plate was removed, and a bisphenol solution was added at 50 μl/well to the plate. The plate was re-set in the real-time cell analyzer, and measurement of electrical resistance values was resumed.

The study consisted of three groups: a bisphenol-free group, a bisphenol-added group (Compound A not added), and a bisphenol-added/Compound A-added group (example group). At the time of bisphenol addition, the final concentration of Compound A in the wells was set to 0 μmol/L (Compound A not added), 0.01 μmol/L, 0.1 μmol/L, or 1 μmol/L, and the final concentration of bisphenol was set to 0 μmol/L (bisphenol not added) or 4 μmol/L.

Then, a normalized electrical resistance value (normalized Cell Index; nCi) 60 hours after bisphenol addition was used as an indicator of suppression of epidermal intercellular adhesion disorder. The nCi was normalized such that the electrical resistance value (Cell Index) immediately before the addition of bisphenol was 1.

### Statistical Analysis

Statistical analysis was performed using SAS (Statistical Analysis System). Comparison between the bisphenol-free group and the bisphenol-added group was conducted using Student's t-test. Further, the pharmacological effect of Compound A was determined by a comparison between the bisphenol-added group and the bisphenol-added/Compound A-added group, using the Williams multiple comparison test as the test method.

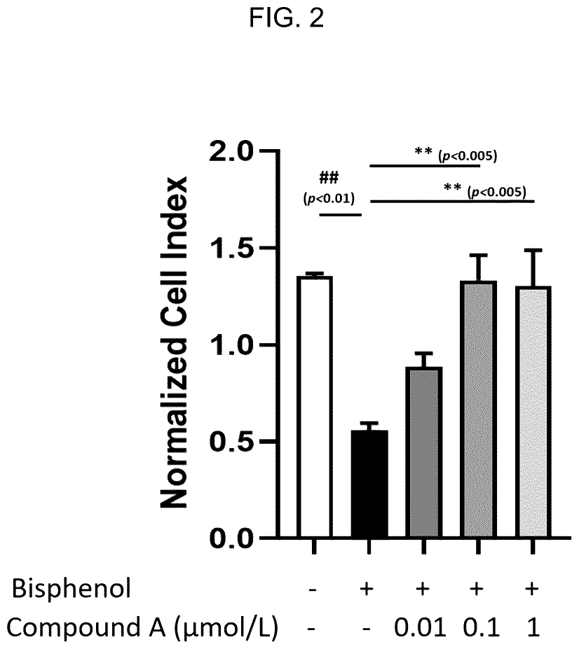

Compound A was evaluated in the in vitro epidermal intercellular adhesion disorder model induced by bisphenol. The above-described test was conducted four times, and average values and standard errors at 60 hours after bisphenol addition were calculated and statistically analyzed. These results are shown in FIG. 2.

FIG. 2 is a graph showing the relationship between the administration of the MPBS monohydrate and the intercellular adhesion in the in vitro epidermal intercellular adhesion disorder model, with the vertical axis representing the normalized electrical resistance value. As shown in FIG. 2, in the bisphenol-added group, where bisphenol was added to the cultured human primary keratinocytes under a final calcium concentration of 1.2 mmol/L, the nCi was statistically significantly decreased compared to the bisphenol-free group (##p<0.01). Then, compared to the bisphenol-added group, the bisphenol-added/Compound A-added group, which was pretreated with Compound A, was found to restore the nCi that had decreased due to bisphenol addition. In particular, pretreatment with Compound A at concentrations of 0.1 to 1 μmol/L resulted in a statistically significant recovery of the nCi that had decreased due to bisphenol addition (** p<0.005). These results suggest that Compound A has an improvement effect on bisphenol-induced epidermal intercellular adhesion disorder.

### Example 3 Transcriptome Analysis in an In Vitro Epidermal Intercellular Adhesion Disorder Model

The effect of Compound A on the transcriptome, that is, the expression changes of gene transcription products, in the epidermal intercellular adhesion disorder model, was comprehensively evaluated. Gene expression changes were evaluated using the sequencer NextSeq500/550, based on variations in RNA count values or variations in RNA expression levels obtained by normalizing the RNAcount values.

### Test Design and Data Analysis Method

Except as described below, the culture was performed under the same conditions as in Example 2 to prepare the epidermal intercellular adhesion disorder model.

Experimental groups (1) to (3) of the present example are described below.

### (1) Group 1 (Control Group)

On Day 1, calcium was added to the wells, and the cells were cultured for 75 hours in the presence of calcium at a final concentration of 1.2 mmol/L. This experimental group was not subjected to bisphenol addition treatment and was designed to simulate normal keratinocytes in the presence of calcium.

### (2) Group 2 (Bisphenol-Added Group)

On Day 1, calcium was added to the wells, and the cells were cultured for 24 hours in the presence of calcium at a final concentration of 1.2 mmol/L, and then a DMSO solution was added. After culturing for 3 hours following the DMSO addition, bisphenol (final concentration of 4 μmol/L) was further added, and culturing was continued for 48 hours. This experimental group was exposed to bisphenol and designed to simulate the conditions under which HHD occurs.

### (3) Group 3 (Compound A-Added Group)

On Day 1, calcium was added to the wells, and the cells were cultured for 24 hours in the presence of calcium at a final concentration of 1.2 mmol/L, and then, Compound A (final concentration of 1 μmol/L) was added. After culturing for 3 hours following the Compound A addition, bisphenol (final concentration of 4 μmol/L) was further added, and culturing was continued for 48 hours. This experimental group was designed to simulate the conditions of administering Compound A to subjects with HHD.

After completion of the culture, cells were collected from each of the experimental groups (1) to (3), and total RNA was extracted using RNeasy (registered trademark) Mini QIAcube columns (Qiagen) according to the product protocol. All extracted RNA samples were subjected to DNase treatment. After that, using NanoDrop (Thermo Fisher Scientific) and Bioanalyzer RNA6000 Nano Chip (Agilent), it was confirmed that all of the extracted RNA samples had an RIN value of 9.4 or higher and a total RNA amount of 1.3 μg or more, and were of high quality.

Using the extracted total RNA (10 ng) as a template, cDNA libraries were prepared using NEBNext (registered trademark) Ultra II Directional RNA Library prep for Illumina (NEB), NEBNext (registered trademark) Poly (A) mRNA Magnetic Isolation Module (NEB), and NEBNext (registered trademark) Multiplex Oligos for Illumina (96 Unique Dual Index Primer Pairs, NEB), according to the product protocols.

Next, the sequences of the cDNA libraries were determined using the NextSeq500/550 system (Illumina) according to the product protocol. Quality control was performed using FASTQC (version 0.12.1) and Trimmomatic-0.39, and the data were converted into FASTQ files.

Subsequently, using StrandNGS software (version 4.0, Strand Life Sciences) and Star (version 2.7.10b), gene types were identified from the 150-base sequence information contained in the FASTQ files, referencing the human genome assembly and transcript annotation (Release 101) obtained from the Ensembl database. Then, the expression levels of these transcripts were calculated. The expression levels of the transcripts were determined using read count values and normalized TPM values.

Differential expression analysis was performed using DESeq2 (a two-group comparison package) in the statistical analysis software R (version 4.3.2) to calculate the log 2 ratio of RNA expression levels and conduct significance testing. The significance testing was performed based on p-values calculated using the Wald test with multiple testing correction based on the Benjamini-Hochberg method (BH method). In the present analysis, a gene with an absolute log 2 ratio of 0.585 or greater (expression change ratio of 1.5-fold or greater) or 0.263 or greater (expression change ratio of 1.2-fold or greater), and a p-value of less than 0.1 from the Wald test between any two groups, was identified as a “gene with a change in expression level” (differentially expressed gene; DEG). DEGs include both upregulated genes and downregulated genes.

Enrichment analysis for gene ontology was conducted by comparing ontology terms defined as Biological Process in the Gene Ontology database, the gene information associated with these ontology terms, and the DEGs. Then, ontology terms with p-values less than 0.05, calculated using Fisher's exact test with multiple testing correction based on the BH method, were considered significant, and annotations were performed for the DEGs. As a result, ontology terms related to biological functions associated with the DEGs were identified.

The expression levels of the transcripts in the experimental groups were compared. As a result, in the comparison between Group 1 and Group 2, 4,827 genes were identified as DEGs (based on a log 2 ratio threshold of 0.585 or greater, with 2,025 genes upregulated and 2,802 genes downregulated in Group 2, the pathological group). Further, in the comparison between Group 2 and Group 3, 1,515 genes were identified as DEGs (based on a log 2 ratio threshold of 0.263 or greater, with 538 genes upregulated and 977 genes downregulated in Group 3, the drug-treated).

The results of the enrichment analysis are shown in Tables 2 and 3. Table 2 shows the biological function ontology terms associated with DEGs that were upregulated by bisphenol and downregulated by Compound A, while Table 3 shows the biological function ontology terms associated with DEGs that were downregulated by bisphenol and upregulated by Compound A. Among the ontology terms related to biological functions associated with the obtained DEGs, 6 ontology terms were identified that were associated with DEGs upregulated in the bisphenol-added group (Group 2; pathological group), but conversely associated with DEGs downregulated in the Compound A-added group (Group 3) (Table 2). Further, 10 ontology terms were identified that were associated with DEGs downregulated in the bisphenol-added group (Group 2; pathological group), but conversely associated with DEGs upregulated in the Compound A-added group (Group 3) (Table 3). These results suggest that bisphenol addition induces activation of endoplasmic reticulum (ER) stress and changes in Golgi apparatus function, and that Compound A has a suppressive effect against these changes. Further, it was suggested that bisphenol addition induces suppression of epidermal differentiation and weakening of intercellular adhesion, and that Compound A normalizes these changes. These results suggest that Compound A has an improvement effect on bisphenol-induced epidermal intercellular adhesion disorder.

TABLE 2

| | Bisphenol change Group | | Compound A change Group | |
|---|---|---|---|---|
| | 1 vs Group 2 comparison | | 2 vs Group 3 comparison | |
| | Direction | Adjusted | Direction | Adjusted |
| Ontology terms associated with DEGs | of change | P-value | of change | P-value |
| Response to endoplasmic reticulum | UP | 3.708E−07 | DOWN | 3.576E−08 |
| stress | | | | |
| Response to topologically incorrect | UP | 1.311E−04 | DOWN | 1.617E−05 |
| protein | | | | |
| Golgi vesicle transport | UP | 1.164E−03 | DOWN | 6.740E−04 |
| Regulation of response to endoplasmic | UP | 1.604E−03 | DOWN | 7.435E−03 |
| reticulum stress | | | | |
| Intrinsic apoptotic signaling pathway | UP | 2.497E−03 | DOWN | 7.253E−03 |
| in response to endoplasmic reticulum | | | | |
| stress | | | | |
| Cellular response to topologically | UP | 9.209E−03 | DOWN | 1.544E−02 |
| incorrect protein | | | | |

TABLE 3

| | Bisphenol change Group | | Compound A change Group | |
|---|---|---|---|---|
| | 1 vs Group 2 comparison | | 2 vs Group 3 comparison | |
| | Direction | Adjusted | Direction | Adjusted |
| Ontology terms associated with DEGs | of change | P-value | of change | P-value |
| Alcohol metabolic process | DOWN | 9.393E−12 | UP | 1.749E−05 |
| Skin development | DOWN | 2.379E−08 | UP | 2.729E−32 |
| Sterol biosynthetic process | DOWN | 1.873E−07 | UP | 6.905E−04 |
| Epidermis development | DOWN | 1.783E−06 | UP | 1.825E−32 |
| Keratinocyte differentiation | DOWN | 2.876E−05 | UP | 2.557E−32 |
| Skin epidermis development | DOWN | 1.894E−04 | UP | 9.388E−04 |
| Olefinic compound metabolic process | DOWN | 9.998E−04 | UP | 1.427E−03 |
| Peptide cross-linking | DOWN | 1.525E−03 | UP | 1.331E−08 |
| Regulation of peptidase activity | DOWN | 1.462E−02 | UP | 2.016E−04 |
| Establishment of skin barrier | DOWN | 4.822E−02 | UP | 2.016E−04 |

According to a pharmaceutical composition according to an embodiment of the present invention, by containing 5-methyl-2-(1-piperazinyl)benzenesulfonic acid. Hailey-Hailey disease can be treated or prevented.

Hailey-Hailey disease is a type of skin disease in which blister-like eruptions (vesicles) frequently appear in intertriginous areas such as the neck, armpits, and groin due to acantholysis of the epidermis. The blisters are accompanied by itching and, when severe, may rupture, resulting in an erosive state with exposed mucous membranes. These conditions can be accompanied by crusts, pustules, pigmentation, or secondary infections, forming lesions resembling impetigo and producing a foul odor. Further, since the disease is prone to chronicity and recurrence, it is accompanied by a decline in patients' quality of life (hereinafter also referred to as QOL), and social handicap is also a concern.

Hailey-Hailey disease currently lacks a fundamental treatment method recommended by guidelines, and treatment is centered on disease management and symptom control by avoiding exacerbating factors. For example, patients may receive lifestyle guidance to reduce factors that worsen symptoms, such as friction, sweating, and sun exposure. In the mild cases, treatment is performed by administering steroid ointments or vitamin D preparations or the like, and in severe cases, treatment may involve administration of immunosuppressants or retinoids or the like. Further, laser therapy or surgical debridement may also be performed.

Treatments involving retinoids, steroids, vitamin D preparations, and the like have been reported to have side effects, and no treatment provides safe and consistently effective results. Therefore, there remains a significant unmet medical need for safe and effective treatments for Hailey-Hailey disease. The entire contents of this publication are incorporated herein by reference.

A pharmaceutical composition for Hailey-Hailey disease according to an embodiment of the present invention contains 5-methyl-2-(1-piperazinyl)benzenesulfonic acid (5-methyl-2-(piperazin-1-yl)benzenesulfonic acid).

A method for treating or preventing Hailey-Hailey disease according to an embodiment of the present invention includes a process of administering 5-methyl-2-(1-piperazinyl)benzenesulfonic acid to a subject.

An embodiment of the present invention is 5-methyl-2-(1-piperazinyl)benzenesulfonic acid for use in treating or preventing Hailey-Hailey disease.

An embodiment of the present invention is 5-methyl-2-(1-piperazinyl)benzenesulfonic acid in manufacturing a pharmaceutical composition for Hailey-Hailey disease.

According to a pharmaceutical composition according to an embodiment of the present invention, by containing 5-methyl-2-(1-piperazinyl)benzenesulfonic acid, Hailey-Hailey disease can be treated or prevented.

A pharmaceutical composition for Hailey-Hailey disease according to an embodiment of the present invention includes 5-methyl-2-(1-piperazinyl)benzenesulfonic acid.

In the pharmaceutical composition, the 5-methyl-2-(1-piperazinyl)benzenesulfonic acid may be an anhydrate thereof, a salt thereof, a hydrate or solvate thereof, or a hydrate or solvate of a salt thereof.

In the pharmaceutical composition, the 5-methyl-2-(1-piperazinyl)benzenesulfonic acid may be an anhydrate thereof, a salt thereof, a hydrate thereof, or a hydrate of a salt thereof.

In the pharmaceutical composition, the hydrate ma be 5-methyl-2-(1-piperazinyl)benzenesulfonic acid monohydrate.

The pharmaceutical composition may be for suppressing acantholysis in the Hailey-Hailey disease.

The pharmaceutical composition may be for improving IGA score in the Hailey-Hailey disease.

The pharmaceutical composition may be for suppressing or improving at least one selected from a group of lesion skin area, itching, pain, and odor in the Hailey-Hailey disease.

In the pharmaceutical composition, dosage of 5-methyl-2-(1-piperazinyl)benzenesulfonic acid is 1 to 1000 mg/day. The dosage is calculated as anhydride of 5-methyl-2-(1-piperazinyl)benzenesulfonic acid.

A method for treating or preventing Hailey-Hailey disease according to an embodiment of the present invention includes a process of administering 5-methyl-2-(1-piperazinyl)benzenesulfonic acid to a subject.

In the method, the 5-methyl-2-(1-piperazinyl)benzenesulfonic acid may be an anhydrate thereof, a salt thereof, a hydrate or solvate thereof, or a hydrate or solvate of a salt thereof.

In the method, the 5-methyl-2-(1-piperazinyl)benzenesulfonic acid may be an anhydrate thereof, a salt thereof, a hydrate thereof, or a hydrate of a salt thereof.

In the method, the hydrate may be 5-methyl-2-(1-piperazinyl)benzenesulfonic acid monohydrate.

In the method, an administration method is oral administration.

In the method, a dose per day may be 1 to 1000 mg/day. The dose is an amount calculated as anhydrate of 5-methyl-2-(1-piperazinyl)benzenesulfonic acid.

In the method, the number of administrations per day may be 1 to 3.

In the method, administration interval may be daily administration.

An embodiment of the present invention is 5-methyl-2-(1-piperazinyl)benzenesulfonic acid for use in treating or preventing Hailey-Hailey disease.

The 5-methyl-2-(1-piperazinyl)benzenesulfonic acid may be an anhydrate thereof, a salt thereof, a hydrate or solvate thereof, or a hydrate or solvate of a salt thereof.

The 5-methyl-2-(1-piperazinyl)benzenesulfonic acid may be an anhydrate thereof, a salt thereof, a hydrate thereof, or a hydrate of a salt thereof.

In the 5-methyl-2-(1-piperazinyl)benzenesulfonic acid, the hydrate may be 5-methyl-2-(1-piperazinyl)benzenesulfonic acid monohydrate.

An embodiment of the present invention may be 5-methyl-2-(1-piperazinyl)benzenesulfonic acid in manufacturing a pharmaceutical composition for Hailey-Hailey disease.

The 5-methyl-2-(1-piperazinyl)benzenesulfonic acid may be an anhydrate thereof, a salt thereof, a hydrate or solvate thereof, or a hydrate or solvate of a salt thereof.

The 5-methyl-2-(1-piperazinyl)benzenesulfonic acid may be an anhydrate thereof, a salt thereof, a hydrate thereof, or a hydrate of a salt thereof.

In the 5-methyl-2-(1-piperazinyl)benzenesulfonic acid, the hydrate may be 5-methyl-2-(1-piperazinyl)benzenesulfonic acid monohydrate.

A pharmaceutical composition for acantholysis according to an embodiment of the present invention includes 5-methyl-2-(1-piperazinyl)benzenesulfonic acid.

In the pharmaceutical composition, the 5-methyl-2-(1-piperazinyl)benzenesulfonic acid may be an anhydrate thereof, a salt thereof, a hydrate or solvate thereof, or a hydrate or solvate of a salt thereof.

In the pharmaceutical composition, the 5-methyl-2-(1-piperazinyl)benzenesulfonic acid may be an anhydrate thereof, a salt thereof, a hydrate thereof, or a hydrate of a salt thereof.

In the pharmaceutical composition, the hydrate may be 5-methyl-2-(1-piperazinyl)benzenesulfonic acid monohydrate.

Obviously, numerous modifications and variations of the present invention are possible in light of the above teachings. It is therefore to be understood that within the scope of the appended claims, the invention may be practiced otherwise than as specifically described herein.

1. A pharmaceutical composition for Hailey-Hailey disease, comprising:
5-methyl-2-(1-piperazinyl)benzenesulfonic acid.

2. A method for treating Hailey-Hailey disease, comprising:
administering a pharmaceutical composition comprising 5-methyl-2-(1-piperazinyl)benzenesulfonic acid to a subject in need thereof.

3. The method of claim 2, wherein the pharmaceutical composition is administered to the subject in need thereof such that a dosage of 5-methyl-2-(1-piperazinyl)benzenesulfonic acid is in a range of 1 to 1000 mg/day.

4. A pharmaceutical composition for acantholysis in Hailey-Hailey disease, comprising:
5-methyl-2-(1-piperazinyl)benzenesulfonic acid.

5. The method of claim 2, wherein the pharmaceutical composition is administered to the subject in need thereof such that at least one of dyskeratosis and acantholysis in Hailey-Hailey disease is suppressed.

6. The method of claim 2, wherein the pharmaceutical composition is administered to the subject in need thereof such that acantholysis in Hailey-Hailey disease is suppressed.

7. The method of claim 2, wherein the pharmaceutical composition is administered to the subject in need thereof such that dyskeratosis in Hailey-Hailey disease is suppressed.

8. The method of claim 2, wherein the subject in need thereof is a patient in need thereof.

9. The method of claim 2, wherein the pharmaceutical composition is orally administered to the subject in need thereof.

10. The method of claim 2, wherein the pharmaceutical composition is parenterally administered to the subject in need thereof.

11. The method of claim 2, wherein the pharmaceutical composition is administered daily to the subject in need thereof.

12. The method of claim 2, wherein the pharmaceutical composition is administered to the subject in need thereof in a range of 1 to 3 times per day.

13. The method of claim 2, wherein the pharmaceutical composition is administered to the subject in need thereof such that a dosage of the 5-methyl-2-(1-piperazinyl)benzenesulfonic acid is in a range of 10 to 500 mg/day.

14. The method of claim 2, wherein the pharmaceutical composition is administered to the subject in need thereof such that a dosage of the 5-methyl-2-(1-piperazinyl)benzenesulfonic acid is in a range of 100 to 400 mg/day.

15. The method of claim 3, wherein the pharmaceutical composition is administered to the subject in need thereof such that at least one of dyskeratosis and acantholysis in Hailey-Hailey disease is suppressed.

16. The method of claim 3, wherein the pharmaceutical composition is administered to the subject in need thereof such that acantholysis in Hailey-Hailey disease is suppressed.

17. The method of claim 3, wherein the pharmaceutical composition is administered to the subject in need thereof such that dyskeratosis in Hailey-Hailey disease is suppressed.

18. The method of claim 3, wherein the subject in need thereof is a patient in need thereof.

19. The method of claim 3, wherein the pharmaceutical composition is orally administered to the subject in need thereof.

20. The method of claim 3, wherein the pharmaceutical composition is parenterally administered to the subject in need thereof.
